(19) United States
(12) Patent Application Publication
(10) Pub. No.: US 2014/0150528 A1
(43) Pub. Date: Jun. 5, 2014

(54) Simplified method for measuring concentration of various exhaust gas mixture components utilizing dissimilar sensors

(71) Applicants: Ronald S. Patrick, Mountain View, CA (US); Robert Lupul, Mountain View, CA (US)

(72) Inventors: Ronald S. Patrick, Mountain View, CA (US); Robert Lupul, Mountain View, CA (US)

(73) Assignee: Engine Control and Monitoring, Los Altos, CA (US)

(21) Appl. No.: 13/815,148

(22) Filed: Feb. 4, 2013

Related U.S. Application Data
(60) Provisional application No. 61/797,133, filed on Nov. 30, 2012.

(51) Int. Cl. G01N 33/00

(52) U.S. Cl. CPC G01N 33/0004
USPC 73/23.31

(57) ABSTRACT

A simplified method for measuring a first property and a second property of an exhaust gas mixture utilizing sensors manufactured for the purpose of measuring a first property, but having a cross-sensitivity to a second property of the exhaust gas mixture. Sensor response characteristics to the first property and the second property are accounted for and used in conjunction with a direct differential measurement between sensors to quantify the concentrations of a first and a second property.

### CROSS-REFERANCE TO RELATED APPLICATIONS

The present application is submitted with reference to, and claims the benefit of, provisional patent application US 61/797,133 filed on Nov. 30, 2012. The title of the cited provisional application is “Simplified method for measuring concentrations of various exhaust gas mixture components utilizing dissimilar sensors”. The text of the first sentence following the title of the specification of the cited provisional patent application is “A simplified method for measuring a first property and a second property of an exhaust gas mixture utilizing sensors manufactured for the purpose of measuring a first property, but having a cross-sensitivity to a second property of the exhaust gas mixture.”.

### STATEMENT REGARDING FEDERALLY SPONSERD RESEARCH

(Not Applicable)

### BACKROUND OF THE INVENTION

Current ceramic NOₓ sensors exhibit cross-sensitivities to NH₃. This cross-sensitivity reduces the accuracy of the reported NOₓ concentration from a sensor if NH₃ is also present in the exhaust gas mixture. These NOₓ sensors have a cross-sensitivity to NH₃ greater than 0 but less or equal to 1. For example, a NOₓ sensor with a low cross-sensitivity to NH₃ would have a cross-sensitivity value closer to 0, while a NOₓ sensor with a higher cross-sensitivity to NH₃ would have a value closer to 1. A cross-sensitivity value of “a” means that 1 ppm of NH₃ appears as “a” ppm of NOₓ (0<a≦1). The disclosed invention covers a simplified method for measuring concentrations NOₓ and NH₃ in an exhaust gas mixture using NO sensors of different NH₃ cross-sensitivities. Previous inventions required the use of more than one type of sensor (i.e. NOₓ and NH₃ sensors), or other catalytic components. One example of recent prior art (U.S. Pat. No. 7,810,313) simplifies the approach by using at least two sensors in a system, but still requires complex algorithms and a decoupling observer module in order to quantify the relative concentrations of NOₓ and NH₃ in an exhaust gas mixture. The complexity of the above methods is unnecessary and can be reduced further in the non-obvious method of the disclosed invention.

### BRIEF SUMMARY OF THE INVENTION

The disclosed invention covers a simplified method for measuring concentrations NOₓ and NH₃ in an exhaust gas mixture using NOₓ sensors of substantially different NH₃ cross-sensitivities.

### BRIEF DESCRIPTION OF THE DRAWING

(Not Applicable)

### DETAILED DESCRIPTION OF THE INVENTION

Two NOₓ sensors having different, but known, cross-sensitivities to NH₃ are used to determine both NOₓ and NH₃ concentrations using the following method: A difference in signals from a first NOₓ sensor (NOₓ1) with a known cross-sensitivity to NH₃ (a₁) and a second NOₓ sensor (N_{O}2), with a different known cross-sensitivity to NH₃ (a₂), is determined (NOₓ1-NOₓ2). The resulting value can divided by the difference in the respective NH₃ cross-sensitivities of a₁ and a₂ resulting in an accurate NH₃ measurement. The formula is:

NH₃=(NOₓ1−NOₓ2)/(a₁−a₂)

The value for NOₓ concentration can then be determined by multiplying a₁ by NH₃ and subtracting the result from NOₓ1. Two variations of the formula are:

NOₓ=NOₓ1−a₁×NH₃

or

NOₓ=NOₓ2−a₂×NH₃

This process is substantially different from the methods using a decoupling observer module in that the math above can be performed largely, or in whole, as the function of an analog circuit and does not require complex algorithms or processing hardware to achieve a result.

1. A method for simultaneously measuring a plurality of properties associated with an exhaust gas mixture, said method comprising: combining a plurality of sensors wherein each sensor among said plurality of sensors exhibits known sensitivities to a first property and exhibits known, but measurably different, cross-sensitivities to a second property in an exhaust gas mixture; wherein said plurality of sensors provide differential signals indicative of an amount of said first property and said second property in said exhaust gas mixture; directly reading, between two of said sensors, a differential value.

2. The method of claim 1, wherein said plurality of sensors comprises at least two NOₓ sensors, of measurably different cross-sensitivities to NH₃, for detecting. said first property, wherein said first property comprises a concentration of NOₓ; said second property comprises a concentration of NH₃ in said exhaust gas mixture.

3. A method for simultaneously measuring both the NOₓ and NH₃ quantities in an exhaust gas mixture comprising; a first NOₓ sensor exhibiting a first known cross-sensitivity to NH₃; a second NOₓ sensor with a second known cross-sensitivity to NH₃; said first known cross-sensitivity being measurably different from said second known cross-sensitivity; directly measuring a differential between said first NOₓ sensor and said second NOₓ sensor a difference indicative of the quantities of NOₓ and NH₃ in said exhaust gas mixture.

4. The method of claim 1 wherein said first property comprises NOₓ and said second property comprises NH₃.

5. The method of claim 2 further comprising: configuring at least one of said NOₓ sensors to comprise a zirconia-based multilayer sensing element.

6. The method of claim 1 wherein at least one of said sensors among said plurality of sensors comprises an electrically-based sensor.
